(19) United States
(12) Patent Application Publication
(10) Pub. No.: US 2010/0016371 A1
(43) Pub. Date: Jan. 21, 2010

(54) Indole Compounds

(75) Inventors: Gerard Martin Paul Giblin, Essex (GB); Adrian Hall, Essex (GB); David Nigel Hurst, Essex (GB); Tiziana Scoccitti, Essex (GB); Pamela Joan Theobald, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited, Greenford, Middlesex (GB)

(21) Appl. No.: 12/373,307

(22) PCT Filed: Jul. 9, 2007
(86) PCT No.: PCT/EP07/56936

§ 371 Date: Jan. 12, 2009

(30) Foreign Application Priority Data
Jul. 14, 2006 (GB) 0614068.5

(51) Int. Cl. A61K 31/427; C07D 417/04; C07D 401/04; A61K 31/4439; A61P 25/00

(52) U.S. Cl. 514/339; 548/181; 546/277.4; 514/365

(57) ABSTRACT

The present application relates to compounds of formula (I) or a pharmaceutically acceptable derivative thereof; wherein X, R¹, R², and R³ are as defined in the specification, a process for the preparation of such compounds, pharmaceutical compositions comprising such compounds and the use of such compounds in medicine.

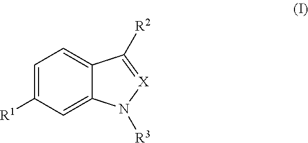

This invention relates to indole compounds, to processes for their preparation, to pharmaceutical compositions containing them and to their use in medicine, in particular their use in the treatment of conditions mediated by the action of PGE₂at the EP₁ receptor.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion. We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996,14, 83-87 and Prostanoid Receptors, Structure, Properties and Function, S Narumiya et al, Physiological Reviews 1999, 79(4),1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, inhibit hormone-induced uterine contractions and have anti-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

In The American Physiological Society (1994, 267, R289-R-294), studies suggest that PGE₂-induced hyperthermia in the rat is mediated predominantly through the EP₁ receptor.

WO 96/06822 (7 Mar. 1996), WO 96/11902 (25 Apr. 1996), EP 752421-A1 (8 Jan. 1997), WO 01/19814 (22 Mar. 2001), WO 03/084917 (16 Oct. 2003), WO 03/101959 (11 Dec. 2003), WO 2004/039753 (13 May 2004), WO 2004/083185 (30 Sep. 2004), WO 2005/037786 (28 Apr. 2005), WO 2005/037793 (28 Apr. 2005), WO 2005/037794 (28 Apr. 2005), WO 2005/040128 (6 May 2005), WO 2005/054191 (16 Jun. 2005), WO2005/108369 (17 Nov. 2005), WO 2006/066968 (29 Jun. 2006), WO 2006/114272 (2 Nov. 2006), WO 2006/114274 (2 Nov. 2006) and WO 2006/114313 (2 Nov. 2006) disclose compounds as being useful in the treatment of prostaglandin mediated diseases.

P. Lacombe et al (220th National Meeting of The American Chemical Society, Washington D.C., USA, 20-24 Aug., 2000) disclosed 2,3-diarylthiophenes as ligands for the human EP₁ prostanoid receptor. Y. Ducharme et al (18^{th} International Symposium on Medicinal Chemistry; Copenhagen, Denmark and Malmo, Sweden; 15-19 Aug. 2004) disclosed 2,3-diarylthiophenes as EP₁ receptor antagonists. Y. Ducharme et al, Biorg. Med. Chem. Lett., 2005, 15(4): 1155 also discloses 2,3-diarylthiophenes as selective EP₁ receptor antagonists.

S. C. McKeown et al, Bioorg. Med. Chem. Lett., 2007, 17, 1750; A. Hall et al, Bioorg. Med. Chem. Lett., 2007, 17, 1200; A. Hall et al, Bioorg. Med. Chem. Lett., 2007, 17, 916; A. Hall et al, Bioorg. Med. Chem. Lett., 2007, 17, 732; G. M. P. Giblin et al, Bioorg. Med. Chem. Lett., 2007, 17, 385-389; S. C. McKeown et al, Bioorg. Med. Chem. Lett., 2006, 16 (18), 4767-4771;” A. Hall et al, Bioorg. Med. Chem. Lett., 2006, 16 (14), 3657-3662; and A. Hall et al, Bioorg. Med. Chem. Lett., 2006, 16 (10), 2666-2671 relate to EP₁ receptor antagonist compounds.

It is now suggested that a novel group of indole and indazole derivatives indicated to be useful in treating conditions mediated by the action of PGE₂ at EP₁ receptors. Such conditions include pain, or inflammatory, immunological, bone, neurodegenerative or renal disorders.

Accordingly the present invention provides one or more chemical entities selected from compounds of formula (I):

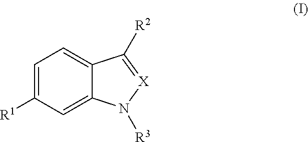

wherein

R¹ represents hydrogen, methyl, —CF₃, chlorine, fluorine or bromine;

R² represents ethyl, propyl, isopropyl, isobutyl, —CH₂-t-butyl, —(CH₂)₂-t-butyl, optionally substituted —CH₂-phenyl, —CH₂—CF₃ or —CO-isopropyl;

X represents CH or N;

R³ represents a group of formula (i)-(ix):

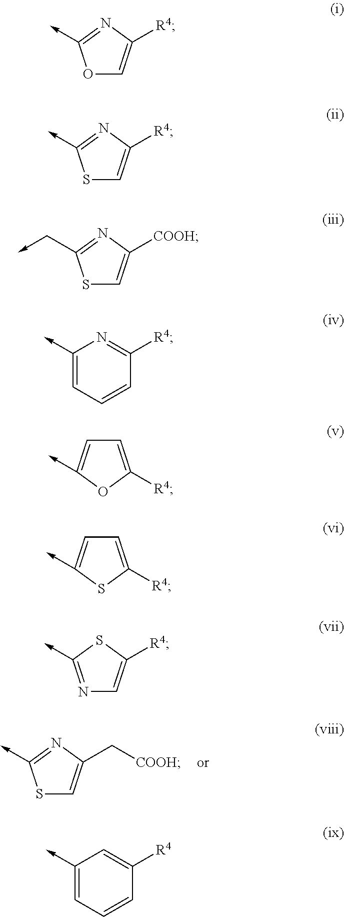

R⁴ represents —COOH, —CO—NH—SO₂—R⁵ or tetrazole;

R⁵ represents C₁₋₃ alkyl, optionally substituted phenyl or 2,4-dimethylisoxazol-4-yl;

such that when R¹ represents hydrogen, R² represents isobutyl;

or derivatives thereof.

Optional substituents for phenyl are selected from optionally substituted C₁₋₆alkyl (e.g. methyl), amino, optionally substituted C₁₋₆alkylamino, hydroxy, HOC₁₋₄alkyl (e.g. HOCH₂) and halogen (e.g. fluorine).

Suitably, R¹ represents bromine or chlorine. In a further embodiment, R¹ represents chlorine.

Suitably, R² represents ethyl, propyl, isopropyl, isobutyl, —CH₂-t-butyl, —(CH₂)₂-t-butyl, benzyl, —CH₂—CF₃ or —CO-isopropyl.

Suitably, X represents CH.

Suitably, R³ represents a group of formula (ii):

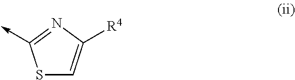

Suitably, R⁴ represents —COOH or —CO—NH—SO₂—R⁵ (e.g. —CO—NH—SO₂-phenyl). In one embodiment, R⁴ represents —COOH.

Compounds of formula (I) include the compounds of Examples 1 to 24 and derivatives thereof.

Particular compounds of formula (I) include the compounds of Examples 4, 5, 6, 11, 12, 13, 17 and 19.

A particular compound is 6-[6-chloro-3-(2-methylpropyl)-1H-indol-1-yl]-2-pyridinecarboxylic acid.

Certain compounds of the Examples are selective for EP₁ over EP₃. Certain compounds of the Examples have greater than 30 fold selectivity.

Derivatives of the compound of formula (I) include salts, solvates (including hydrates), solvates (including hydrates) of salts, esters and polymorphs of the compound of formula (I). Derivatives of the compounds of formula (I) include pharmaceutically acceptable derivatives.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The present invention also includes isotopically-labelled compounds, which are identical to the compounds of formula (I), except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ²H, ³H, ¹¹C, ¹⁴C, ¹⁸F, ³⁵S, ¹²³I and ¹²⁵I.

Compounds of the present invention and pharmaceutically acceptable derivatives (e.g. salts) of said compounds that contain the aforementioned isotopes and/or other isotopes of other atoms are within the scope of the present invention. Isotopically-labelled compounds of the present invention, for example those into which radioactive isotopes such as ³H and/or ¹⁴C are incorporated, are useful in drug and/or substrate tissue distribution assays. ³H and ¹⁴C are considered useful due to their ease of preparation and detectability. ¹¹C and ¹⁸F isotopes are considered useful in PET (positron emission tomography), and ¹²⁵I isotopes are considered useful in SPECT (single photon emission computerized tomography), all useful in brain imaging. Substitution with heavier isotopes such as ²H can afford certain therapeutic advantages resulting from greater metabolic stability, for example increased in vivo half-life or reduced dosage requirements and, hence, are considered useful in some circumstances. Isotopically labelled compounds of formula (I) of this invention can generally be prepared by carrying out the procedures disclosed in the Schemes and/or in the Examples below, by substituting a readily available isotopically labelled reagent for a non-isotopically labelled reagent.

The following definitions are used herein unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I). In one aspect the term “pharmaceutically acceptable derivative” means any pharmaceutically acceptable salt, solvate or solvate of salt. In an alternative aspect the term “pharmaceutically acceptable derivative” means any pharmaceutically acceptable salt.

It will be appreciated that, for pharmaceutical use, the derivatives referred to above will be pharmaceutically acceptable derivatives, but other derivatives may find use, for example in the preparation of compounds of formula (I) and the pharmaceutically acceptable derivatives thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropylamine, tris(hydroxymethyl)aminomethane (TRIS, trometamol) and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

Compounds of formula (I) can be prepared as set forth in the following schemes and in the examples. The following processes form another aspect of the present invention.

Compounds of formula (I) wherein R² represents ethyl, propyl, isobutyl, —CH₂-t-butyl, —(CH₂)₂-t-butyl, optionally substituted —CH₂-phenyl or —CH₂—CF₃, R³ represents a group of formula (ii), X represents CH and R⁴ represents —COOH may be prepared by the general route shown in Scheme 1 below:

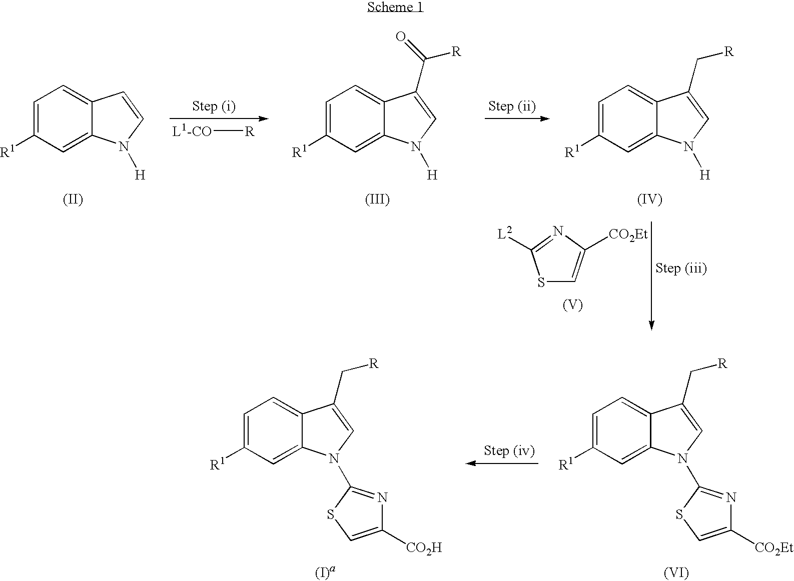

wherein R¹ is as defined above, R represents methyl, ethyl, isopropyl, t-butyl, —CH₂-t-butyl, optionally substituted phenyl or CF₃, L¹ represents a suitable leaving group such as a halogen atom (e.g. chlorine) and L² represents a suitable leaving group such as a halogen atom (e.g. bromine).

Step (i) typically comprises reaction of a compound of formula (II) with a compound of formula L¹-CO—R in the presence of suitable reagents, such as methyl magnesium bromide and zinc chloride.

Step (ii) typically comprises a reduction reaction in the presence of a suitable reducing agent, e.g. lithium aluminium hydride or sodium borohydride.

Step (iii) typically comprises a Buchwald coupling reaction between a compound of formula (IV) and a compound of formula (V) in the presence of a suitable catalyst e.g. copper (I) iodide and a suitable base e.g. potassium phosphate and a suitable amine, in the presence of a suitable solvent e.g. toluene.

Step (iv) typically comprises treatment of a compound of formula (VI) with sodium hydroxide.

Compounds of formula (I) wherein R² is CH₂CF₃ may also be prepared in accordance with the methods as described in the examples.

Compounds of formula (I) wherein R³ is is other than a group of formula (ii) may be prepared by analagous procedures to those described in Scheme 1, for example as described in the Examples.

Compounds of formula (I) wherein R² represents isopropyl, R³ represents a group of formula (ii) and R⁴ represents —COOH may be prepared by the general route shown in Scheme 2 below:

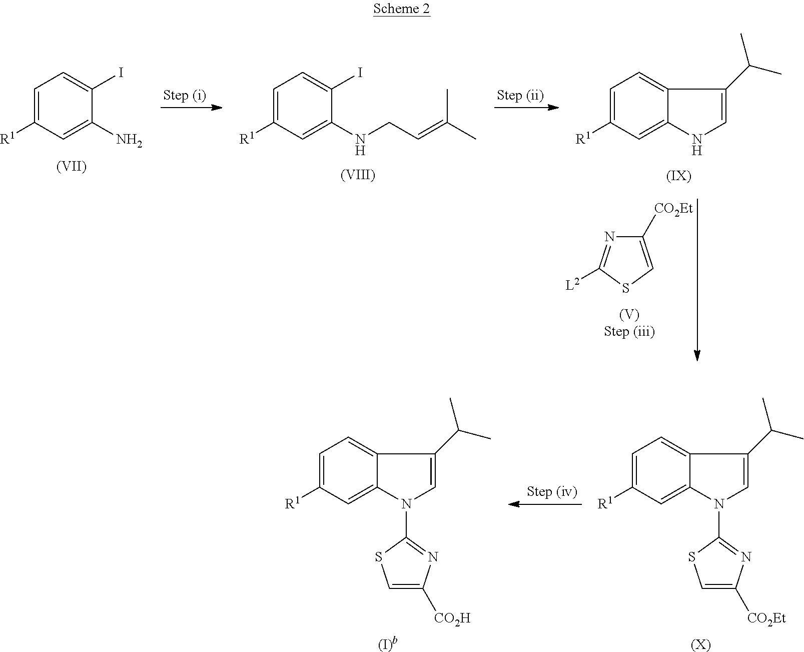

wherein R¹ and L² are as defined above.

Step (i) typically comprises reaction of a compound of formula (VII) with a suitable alkylating reagent, such as 1-bromo-3-methyl but-2-ene, in the presence of a suitable base e.g. lithium diisopropylamide.

Step (ii) typically comprises an intramolecular Heck coupling reaction in the presence of a suitable catalyst e.g. palladium acetate in a suitable solvent e.g. dimethylformamide.

Steps (iii) and (iv) may be performed in an analogous manner to steps (iii) and (iv) in Scheme 1.

Compounds of formula (I) wherein X represents N, R³ represents a group of formula (ii) and R⁴ represents —COOH may be prepared by the general route shown in Scheme 3 below:

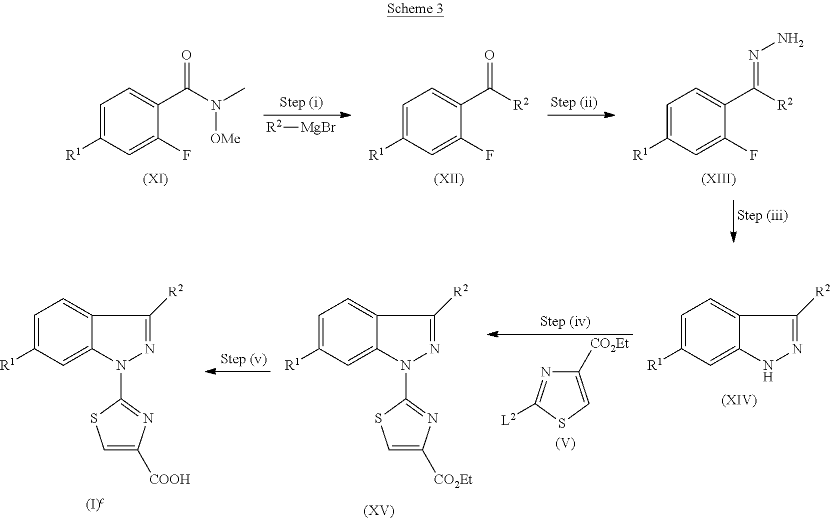

wherein R¹, R² and L² are as defined above.

Step (i) comprises reaction of a compound of formula (XI) with a Grignard reagent of formula R²—MgBr in a suitable solvent e.g. tetrahydrofuran.

Step (ii) comprises reaction of a compound of formula (XII) with hydrazine hydrate in a suitable solvent e.g. ethanol.

Step (iii) comprises intramolecular cyclisation in a suitable solvent e.g. ethylene glycol at an elevated temperature.

Steps (iv) and (v) may be performed in an analogous manner to steps (iii) and (iv) in Schemes 1 and 2.

Compounds of formula (I) wherein R² represents ethyl, propyl, isobutyl, —CH₂-t-butyl, —(CH₂)₂-t-butyl, optionally substituted —CH₂-phenyl or —CH₂—CF₃, R³ represents a group of formula (iii), X represents CH and R⁴ represents —COOH may be prepared by the general route shown in Scheme 4 below:

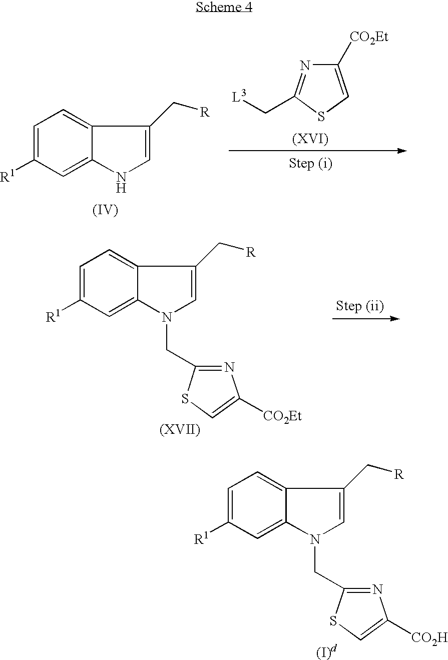

wherein R¹ and R are as defined above and L³ represents a suitable leaving group such as a halogen atom (e.g. bromine).

Step (i) typically comprises treatment of a compound of formula (IV) with a compound of formula (XVI) in the presence of a suitable base e.g. sodium hydride in a suitable solvent e.g. dimethylformamide.

Step (ii) may be performed in an analogous manner to step (iv) in Schemes 1 and 2.

It will be appreciated that compounds of formula (I) wherein R³ represents groups of formulae (i) and (iv) to (ix) may be prepared in an analogous manner to the procedure described in Scheme 1 for compounds wherein R³ represents a group of formula (ii).

It will be appreciated to those skilled in the art that compounds of formula (I) wherein R⁴ represents —CONHSO₂R⁵ or tetrazole may be prepared from compounds of formula (I)^{a} by standard reaction sequences. For example, derivatives wherein R⁴ represents —CONHSO₂R⁵ may be prepared from compounds of formula (I)^{a} by conversion to the acid chloride, for example by reaction with thionyl chloride or oxalyl chloride, in the presence of DMF, in a suitable solvent, such as DCM, followed by reaction with a sulphonamide. Alternative conditions include reaction of a carboxylic acid of formula (I)^{a} with a sulphonamide in a solvent, such as THF or DCM, in the presence of EDC and DMAP. Compounds of formula (I) where R⁴ is tetrazole may be formed from the corresponding carboxylic acid (i.e. compounds of formula (I)^{a}, (I)^{b}, (I)^{c} or (I)^{d}) by converting the carboxylic acid to the primary amide (for example by reaction with sulfonyl chloride followed by ammonia) followed by dehydration of the amide to the nitrile (for example by heating in phosphorous oxychloride) followed by reaction with azide.

Certain substituents in any of the reaction intermediates and compounds of formula (I) may be converted to other substituents by conventional methods known to those skilled in the art. Examples of such transformations include the hydrolysis of esters and esterification of carboxylic acids. Such transformations are well known to those skilled in the art and are described in for example, Richard Larock, Comprehensive Organic Transformations, 2nd edition, Wiley-VCH, ISBN 0-471-19031-4.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

Compounds of formula (II), (V), (VII), (XI) and (XVI) are either commercially available, or may be prepared by known methods.

The compounds of the invention bind to the EP₁ receptor and are antagonists of this receptor. They are therefore considered useful in treating conditions mediated by the action of PGE₂ at EP₁ receptors.

One condition mediated by the action of PGE₂ at EP₁ receptors is pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, multiple sclerosis pain, fibromyalgia, HIV-related neuropathy, post-herpetic neuralgia, trigeminal neuralgia, and pain resulting from physical trauma, amputation, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions mediated by the action of PGE₂ at EP₁ receptors include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) and cyclooxygenase-2 (COX-2) inhibitors, cardiovascular diseases; neurodegenerative diseases and neurodegeneration, neurodegeneration following trauma, tinnitus, dependence on a dependence-inducing agent such as opoids (e.g. morphine), CNS depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence.

Inflammatory conditions include skin conditions (e.g. sunburn, burns, eczema, dermatitis, psoriasis), ophthalmic diseases such as glaucoma, retinitis, retinopathies, uveitis and of acute injury to the eye tissue (e.g. conjunctivitis), inflammatory lung disorders (e.g. asthma, bronchitis, emphysema, allergic rhinitis, respiratory distress syndrome, pigeon fancier's disease, farmer's lung, chronic obstructive pulmonary disease (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

Immunological diseases include autoimmune diseases, immunological deficiency diseases or organ transplantation. The compounds of formula (I) are also effective in increasing the latency of HIV infection.

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

Cardiovascular diseases include hypertension or myocardiac ischemia; functional or organic venous insufficiency; varicose therapy; haemorrhoids; and shock states associated with a marked drop in arterial pressure (e.g. septic shock).

Neurodegenerative diseases include dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, particularly mesangial proliferative glomerulonephritis and nephritic syndrome.

The compounds of formula (I) are also considered useful for the preparation of a drug with diuretic action.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof.

A proposed daily dosage of compounds of formula (I) or their pharmaceutically acceptable derivatives for the treatment of man is from 0.01 to 80 mg/kg body weight, more particularly 0.01 to 30 mg/kg body weight per day, for example 0.1 to 10 mg/kg body weight per day, which may be administered as a single or divided dose, for example one to four times per day. The dose range for adult human beings is generally from 8 to 4000 mg/day, more particularly from 8 to 2000 mg/day, such as from 20 to 1000 mg/day, for example 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 (cyclooxygenase-2 ) inhibitors, such as celecoxib, deracoxib, rofecoxib, valdecoxib, parecoxib, COX-189 or 2-(4-ethoxy-phenyl)-3-(4-methanesulfonyl-phenyl)-pyrazolo[1,5-b]pyridazine (WO99/012930); 5-lipoxygenase inhibitors; NSAIDs (non-steroidal anti-inflammatory drugs) such as diclofenac, indomethacin, nabumetone or ibuprofen; leukotriene receptor antagonists; DMARDs (disease modifying anti-rheumatic drugs) such as methotrexate; adenosine A1 receptor agonists; sodium channel blockers, such as lamotrigine; NMDA (N-methyl-D-aspartate) receptor modulators, such as glycine receptor antagonists; ligands for the α₂δ-subunit of voltage gated calcium channels, such as gabapentin and pregabalin; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands; vanilloid receptor ligand; and purinergic receptor ligands, including antagonists at P2X₃, P2X_{2/3}, P2X₄, P2X₇ or P2X_{4,7}. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. NO. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

No toxicological effects have currently been observed with the compounds of the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

Abbreviations

Solid phase extraction (SPE); liquid chromatography/mass spectrometry (LCMS, LC/MS & LC-MS); MDAP (Mass Directed Auto Preparation); NMR (nuclear magnetic resonance); s, d, t, dd, m, b (singlet, doublet, triplet, doublet of doublets, multiplet, broad); Ph, Me, Et, Pr, Bu, Bn (phenyl, methyl, ethyl, propyl, butyl, benzyl), tetrahydrofuran (THF), dichloromethane (DCM), N,N-dimethylformamide (DMF), h (hours), ethylenediaminetetraacetic acid (EDTA), N-(3dimethylaminopropyl)N′-ethylcarbodiimide hydrochloride (EDC & EDAC), 4-N,N-dimethylaminopyridine (DMAP), ultraviolet (UV), room temperature (RT), retention time (Rt), minutes (min), EtOAc (ethyl acetate), Et₂O (diethyl ether), MeCN (acetonitrile).

Purification of Reaction Products

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the Examples may be carried out by conventional methods such as chromatography and/or recrystallisation using suitable solvents. Chromatographic methods are known to the skilled person and include e.g. column chromatography, flash chromatography, HPLC (high performance liquid chromatography), and MDAP (mass directed autopreparation, also referred to as mass directed LCMS purification). MDAP is described in e.g. W. Goetzinger et al, Int. J. Mass Spectrom., 2004, 238, 153-162.

The terms “Biotage®” and “Flash Master II®” when used herein refer to commercially available automated purification systems using pre-packed silica gel cartridges.

LCMS

The following LCMS conditions were used during the preparation of the examples.

Software

Waters MassLynx version 4.0 SP2

Column

The column used is a Waters Atlantis, the dimensions of which are 4.6 mm×50 mm. The stationary phase particle size is 3 m.

Solvents

A: Aqueous solvent=Water+0.05% Formic Acid

B: Organic solvent=Acetonitrile+0.05% Formic Acid

Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

All retention times are measured in minutes.

### Description 1

### 1-(6-Chloro-1H-indol-3-yl)-2-methyl-1-propanone (D1)

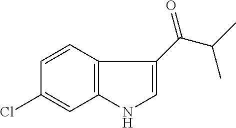

3M Methylmagnesium bromide in ether (1.7 ml, 5.1 mmol) was added to a stirred solution of 6-chloroindole (758 mg, 5 mmol) in dry ether (10 ml) under argon producing a two phase mixture and gas evolution. After stirring for 15 minutes 1 M zinc chloride in ether (5 ml) was added and the mixture stirred for 30 minutes before isobutyryl chloride (533 mg, 5 mmol) was added rapidly with vigorous stirring. The mixture was stirred for 30 minutes and quenched by addition of saturated ammonium chloride solution then diluted with ethyl acetate. The organic phase was dried (magnesium sulphate), evaporated, triturated with ether and filtered to give the title compound as a pink solid (710 mg).

LCMS: Rt=2.89 min, [MH⁺] 222.18, 224.17.

### Descriptions 2-11

The following compounds were prepared in an analogous manner to D1 using indole or a 6-substituted indole and an appropriate chloride:

| Name | Structure | Data |
|---|---|---|
| 1-(6-Chloro-1H-indol-3-yl)-1- propane (D2) | 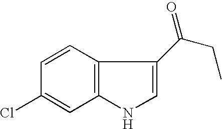 | LCMS Rt = 2.59 min [MH]⁺ 208.18, 210.19 |
| 1-(6-Chloro-1H-indol-3-yl)-3,3- dimethyl-1-butanone (D3) | 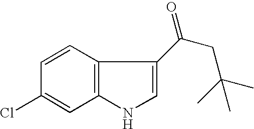 | LCMS Rt = 3.26 min [MH]⁺ 250.27 252.26 |
| (6-Chloro-1H-indol-3- yl)(phenyl)methanone (D4) | 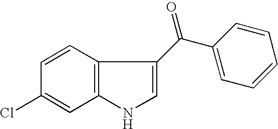 | LCMS Rt = 3.07 min [MH]⁺ 256.18 258.19 |
| 1-(1H-Indol-3-yl)-2-methyl-1- propanone (D5) | 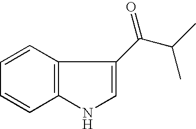 | LCMS Rt = 2.51 min [MH]⁺ 188.2 |
| 1-(6-Bromo-1H-indol-3-yl)-2- methyl-1-propanone (D6) | 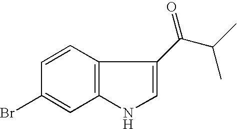 | |
| 1-(6-Chloro-1H-indol-3- yl)ethanone (D7) | 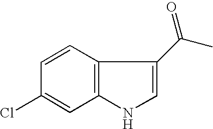 | LCMS Rt = 2.34 min [MH]⁺ 192.11 194.10 |
| 1-(6-Chloro-1H-indol-3-yl)-2,2- dimethyl-1-propanone (D8) | 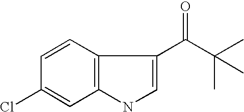 | LCMS Rt = 3.14 min [MH]⁺ 236.1, 238.1 |
| 1-(6-Fluoro-1H-indol-3-yl)-2- methyl-1-propanone (D9) | 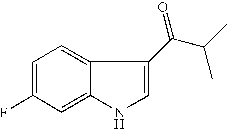 | |
| 2-Methyl-1-[6-(trifluoromethyl)- 1H-indol-3-yl]-1-propanone (D10) | 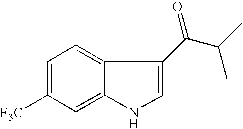 | |

### Description 11

### 6-Chloro-3-(2-methylpropyl)-1H-indole (D11)

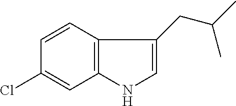

1M Lithium aluminium hydride in THF (7 ml, 7 mmol) was added to a stirred solution of 1-(6-chloro-1H-indol-3-yl)-2-methyl-1-propanone (may be prepared as described in D1; 705 mg, 3.21 mmol) in THF (15 ml) and heated at 55° C. for four hours. The solution was cooled and quenched by careful addition of 2M sodium hydroxide and ether. The organic phase was dried (magnesium sulphate), evaporated and purified on a Biotage column eluting with (1:9) ethyl acetate/hexane to give the title compound as a colourless oil which crystallised on scratching (663 mg).

LCMS: Rt=3.66 min, [MH⁺]208.22, 210.24.

### Descriptions 12-17

The following compounds were prepared by reduction of the appropriate ketone as described in the table below using an analogous procedure to that described in D11:

| | Starting | | |
|---|---|---|---|
| Name | Material | Structure | Data |
| 6-Chloro-3-propyl-1H- indole (D12) | D2 | 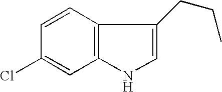 | LCMS Rt = 3.35 min [MH]⁺ 194.23, 196.22 |
| 6-Chloro-3-(3,3- dimethylbutyl)-1H- indole (D13) | D3 | 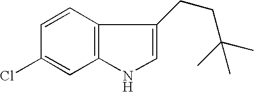 | LCMS Rt = 3.98 min [MH]⁺ 236.26 238.26 |
| 3-(2-Methylpropyl)- 1H-indole (D14) | D5 | 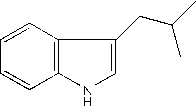 | LCMS Rt = 3.44 min [MH]⁺ 174.1 |
| 6-Chloro-3-ethyl-1H- indole (D15) | D7 | 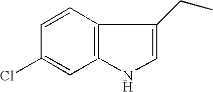 | LCMS Rt = 3.16 min [MH]⁺ 178.06 180.11 |
| 6-Chloro-3- (phenylmethyl)-1H- indole (D16) | D4 | 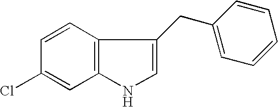 | LCMS Rt = 3.55 min [MH]⁺ 242.27 244.30 |
| 6-Fluoro-3-(2- methylpropyl)-1H- indole (D17) | D9 | 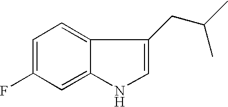 | |

### Description 18

### 6-Chloro-3-(2,2-dimethylpropyl)-1H-indole (D18)

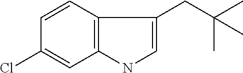

1M Lithium aluminium hydride in THF (7.45 ml, 7.45 mmol) was added to a stirred solution of 1-(6-chloro-1H-indol-3-yl)-2,2-dimethyl-1-propanone (may be prepared as described in D8; 876 mg, 3.72 mmol) in THF (10 ml) and heated at 55° C. for 3 hr. More 1M lithium aluminium hydride in THF was added (3.7 ml) and mixture heated at 55° C. overnight. The solution was cooled and quenched by careful addition of 2M sodium hydroxide and ether. The organic phase was dried, evaporated and purified on a silica column eluting with 15% of ethyl acetate in hexane to give the title compound as oil.

LCMS: Rt=3.84 min, [MH⁻] 220.1, 222.1.

### Description 19

### 3-(2-Methylpropyl)-6-(trifluoromethyl)-1H-indole (D19)

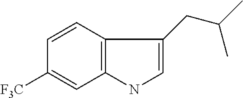

2-Methyl-1-[6-(trifluoromethyl)-1H-indol-3-yl]-1-propanone (may be prepared as described in D10; 2.3 g, 9 mmol) was dissolved in anhydrous THF (30 ml), cooled to 0° C. and treated with 1M borane in THF (21 ml, 21 mmol) dropwise at 0° C. The mixture was stirred at room temperature for 1 hr, cooled in ice, treated with 1.5M hydrochloric acid (15 ml) dropwise, extracted with ethyl acetate, dried over sodium sulphate and evaporated to give the title compound (1.52 g) as a green solid.

### Description 20

### 6-Bromo-3-(2-methylpropyl)-1H-indole (D20)

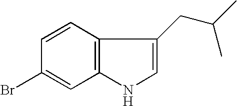

D20 was prepared from D6 in an analogous manner to that described for D19.

### Description 21

### (5-Chloro-2-iodophenyl)(3-methyl-2-buten-1-yl)amine (D21)

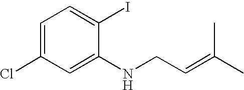

2M Lithium diisopropylamide in heptane/THF/ethylbenzene (4.16 ml, 8.32 mmol) were added to a stirred solution of 5-chloro-2-iodoaniline in dry THF (35 ml) at −78° C. under argon then allowed to warm to 0° C. before being cooled to −78° C. and 1-bromo-3-methylbut-2-ene (1.364 g, 9.15 mmol) was added. The resulting solution was stirred for ten minutes at −78° C. then allowed to warm to room temperature and stirred for one hour before quenching with water/ether. The organic phase was dried (magnesium sulphate), evaporated and purified on a Biotage column eluting with hexane to give the title compound as a pale coloured oil (2.11 g).

LCMS: Rt=4.16 min.

### Description 22

### 6-Chloro-3-(1-methylethyl)-1H-indole (D22)

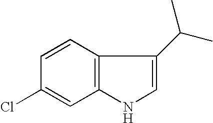

A mixture of (5-chloro-2-iodophenyl)(3-methyl-2-buten-1-yl)amine (may be prepared as described in D21; 2.15 g, 6.7 mmol), palladium acetate (30 mg, 0.134 mmol), tetrabutylammonium bromide (2.157 g, 6.7 mmol) and triethylamine (1.69 g, 16.73 mmol) in dimethylformamide (12 ml) was stirred and heated at 80° C. under argon for one hour. The resulting mixture was cooled, diluted with ether/water and the organic phase was dried (magnesium sulphate), evaporated and purified on a Biotage column eluting with ethyl acetate/hexane (1:99) then recrystallised from hexane to give the title compound as a white solid (710 mg).

LCMS: Rt=3.52 min, [MH⁺] 194.21, 196.18.

### Description 23

### 1-(6-Chloro-1H-indol-3-yl)-2,2,2-trifluoroethanone (D23)

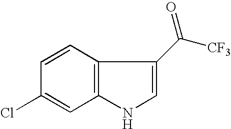

A solution of 6-chloroindole (1.515 g, 10 mmol) in ether (10 ml) was added to an ice cooled solution of trifluoroacetic anhydride (3.5 ml) in ether (30 ml) over 2 minutes and the resulting solution left in the fridge overnight. The resulting suspension was evaporated and the residue triturated with ether and filtered to give the title compound as a sandy coloured solid (2.19 g).

LCMS: Rt=3.12 min, [MH⁺] 248.14, 250.06.

### Description 24

### 1,1-Dimethylethyl 6-chloro-3-(trifluoroacetyl)-1H-indole-1-carboxylate (D24)

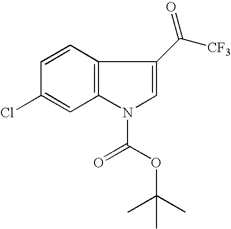

4-Dimethylaminopyridine (2.135 g, 17.5 mmol) was added to a stirred suspension of 1-(6-chloro-1H-indol-3-yl)-2,2,2-trifluoroethanone (may be prepared as described in D23; 4.339, 17.5 mmol) and di-tert-butyl dicarbonate (3.82 g, 17.5 mmol) in dichloromethane (50 ml) producing a clear solution which was left at room temperature for one hour. The solution was washed with 2M hydrochloric acid, dried (magnesium sulphate), evaporated and the residue triturated with ether and filtered to give the title compound as a white solid (4.71 g).

### Description 25

### 1,1-Dimethylethyl 6-chloro-3-(2,2,2-trifluoro-1-hydroxyethyl)-1H-indole-1-carboxylate (D25)

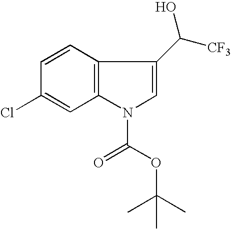

Sodium borohydride (1 g, 26.32 mmol) was added to a stirred suspension of 1,1-dimethylethyl 6-chloro-3-(trifluoroacetyl)-1H-indole-1-carboxylate (may be prepared as described in D24; 4.7 g, 13.53 mmol) in ethanol (60 ml) and stirred for one hour. The resulting solution was evaporated and the residue dissolved in ethyl acetate/water and the organic phase dried (magnesium sulphate) and evaporated to give the title compound as a colourless gum (4.7 g).

LCMS: Rt=3.61 min.

### Description 26

### 1,1-Dimethylethyl 6-chloro-3-(2,2,2-trifluoro-1-{[(phenyloxy)carbonothioyl]oxy}ethyl)-1H-indole-1-carboxylate (D26)

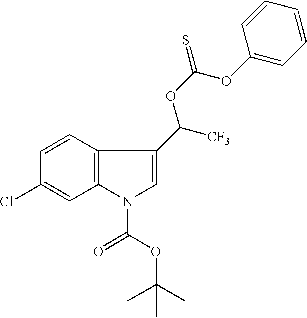

A solution of phenyl chlorothionoformate (173 mg, 1 mmol) in dichloromethane (1 ml) was added to a solution of 1,1-dimethylethyl 6-chloro-3-(2,2,2-trifluoro-1-hydroxyethyl)-1H-indole-1-carboxylate (may be prepared as described in D25; 350 mg, 1 mmol) and 4-dimethylaminopyridine (5 mg) in pyridine (3 ml) and left in the fridge for 3 days. The resulting solution was diluted with ethyl acetate/2M hydrochloric acid and the organic phase dried (magnesium sulphate), evaporated and purified on a Biotage column eluting with dichloromethane/hexane (1:4) to give the title compound as a colourless gum (366 mg).

LCMS: Rt=4.37 min.

### Description 27

### 1,1-Dimethylethyl 6-chloro-3-(2,2,2-trifluoroethyl)-1H-indole-1-carboxylate (D27)

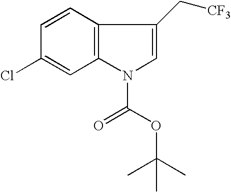

A mixture of 1,1-dimethylethyl 6-chloro-3-(2,2,2-trifluoro-1-{[(phenyloxy)carbonothioyl]oxy}ethyl)-1H-indole-1-carboxylate (may be prepared as described in D26; 350 mg, 0.72 mmol), tributyltin hydride (315 mg, 1.08 mmol) and 2,2′-azobis(2-methylpropionitrile) (23 mg, 0.14 mmol) in toluene (10 ml) was stirred and heated at 80° C. for 3 hours. The solution was cooled, evaporated and purified on a Biotage column eluting with ethyl acetate/hexane (1:49) then on another column eluting with dichloromethane/hexane (1:4) to give the title compound as a gum which crystallised (151 mg).

LCMS: Rt=3.98 min.

### Description 28

### 6-Chloro-3-(2,2,2-trifluoroethyl)-1H-indole (D28)

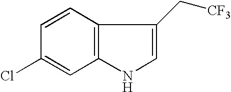

1,1-Dimethylethyl 6-chloro-3-(2,2,2-trifluoroethyl)-1H-indole-1-carboxylate (may be prepared as described in D27; 146 mg, 0.44 mmol) was dissolved in trifluoroacetic acid/dichloromethane (1:1, 3 ml) and left at room temperature for one hour. The solution was evaporated and the residue triturated with hexane and filtered off to give the title compound as a white solid (86 mg).

LC/MS: Rt=3.05 min, [MH⁻] 232.07, 234.06.

### Description 29

### 1-(4-Chloro-2-fluorophenyl)-3-methyl-1-butanone (D29)

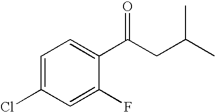

4-Chloro-2-fluoro-N-methyl-N-(methyloxy)benzamide (6.1 g, 28.05 mmol) in THF (20 ml) was added over 10 minutes to isobutylmagnesium bromide (prepared from 800 mg, 32.92 mmol of magnesium and 4.11 g, 30 mmol of isobutyl bromide in 50 ml of THF). Stirred for 30 minutes at room temperature then heated at 60° C. for 4 hours. The solution was cooled, diluted with 2M hydrochloric acid/ether and the organic phase dried (magnesium sulphate), evaporated and purified on a Biotage column eluting with ethyl acetate/hexane (5:195) to give the title compound as colourless oil (806 mg).

LCMS: Rt=3.58 min.

### Description 30

### 1-(4-Chloro-2-fluorophenyl)-3-methyl-1-butanone hydrazone (D30)

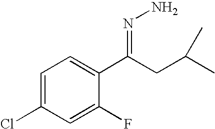

Hydrazine hydrate (400 mg, 8 mmol) was added to a solution of 1-(4-chloro-2-fluorophenyl)-3-methyl-1-butanone (may be prepared as described in D29; 800 mg, 3.73 mmol) in ethanol (10 ml) and left at room temperature overnight. The resulting solution was evaporated, dissolved in ethyl acetate/water and the organic phase dried (magnesium sulphate), evaporated and purified on a Biotage column eluting with ethyl acetate/hexane (15:85) to give the title compound as colourless oil (585 mg).

LCMS: Rt=2.90, 2.97 min [MH⁺] 229.22, 231.21.

### Description 31

### 6-Chloro-3-(2-methylpropyl)-1H-indazole (D31)

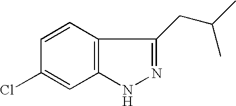

1-(4-Chloro-2-fluorophenyl)-3-methyl-1-butanone hydrazone (may be prepared as described in D30; 580 mg) in ethylene glycol (5 ml) was stirred and heated at 165° C. for 3 hours. The solution was cooled diluted with ether/water and the organic phase washed with water, dried (magnesium sulphate), evaporated and purified on a Biotage column eluting with ethyl acetate/hexane (1:7) to give the title compound as white solid (170 mg).

LCMS: Rt=3.10 min [MH⁺] 209.24, 211.23.

### Description 32

### Ethyl 2-[6-chloro-3-(2-methylpropyl)-1H-indol-1-yl]-1,3-thiazole-4-carboxylate (D32)

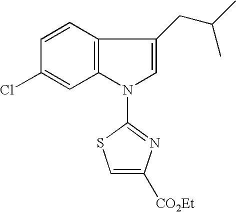

A mixture of 6-chloro-3-(2-methylpropyl)-1H-indole (may be prepared as described in D11; 660 mg, 3.18 mmol), ethyl 2-bromo-4-thiazolecarboxylate (750 mg, 3.18 mmol), potassium phosphate (1.416 g, 6.68 mmol), copper(I) iodide (30 mg, 0.16 mmol) and (1R, 2R)-N,N′-dimethyl-1,2-cyclohexanediamine (53 mg, 0.37 mmol) in toluene (4 ml) was stirred and heated at 110° C. under argon for 48 hours when a further quantity of copper(I) iodide (30 mg, 0.16 mmol) and (1R, 2R)-N,N′-dimethyl-1,2-cyclohexanediamine (53 mg, 0.37 mmol) was added. After heating for a further 24 hours the mixture was cooled, diluted with ether/water and the organic phase dried (magnesium sulphate), evaporated and purified on a Biotage column eluting with (8:92) ethyl acetate/hexane to give the title compound as a white solid (380 mg).

LCMS: Rt=4.14 min, [MH⁺] 363.24, 363.23.

### Descriptions 33-43

The following compounds were prepared in an analogous method to that described for D32 using the appropriate indole or indazole and a bromo heterocycle or a bromobenzene. Ethyl 2-bromo-1,3-oxazole-4-carboxylate was prepared as described in Organic Letters 4(17), 2905-2907 (2002).

| | Starting | | |
|---|---|---|---|
| Name | Material | Structure | Data |
| Ethyl 2-(6-chloro-3- propyl-1H-indol-1-yl)- 1,3-thiazole-4- carboxylate (D33) | D12 | 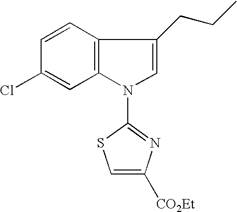 | LCMS Rt = 4.01 min [MH]⁺ 349.22, 351.22 |
| Ethyl 2-[6-chloro-3- (3,3-dimethylbutyl)- 1H-indol-1-yl]-1,3- thiazole-4- carboxylate (D34) | D13 | 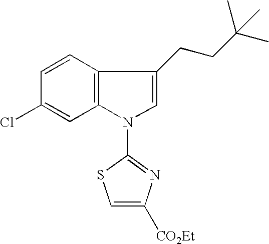 | LCMS Rt = 4.55 min [MH]⁺ 391.24, 393.24 |
| Ethyl 2-[3-(2- methylpropyl)-1H- indol-1-yl]-1,3- thiazole-4- carboxylate (D35) | D14 | 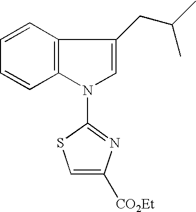 | LCMS Rt = 3.91 min [MH]⁺ 329.30 |
| Ethyl 2-[6-bromo-3- (2-methylpropyl-1H- indol-1-yl]-1,3- thiazole-4- carboxylate (D36) | D20 | 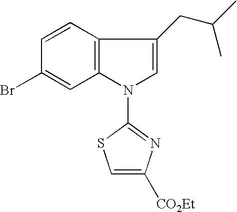 | LCMS Rt = 4.27 min [MH]⁺ 409.14, 410.14 |
| Ethyl 2-[6-chloro-3- (2-methylpropyl)-1H- indol-1-yl]-1,3- oxazole-4- carboxylate (D37) | D11 | 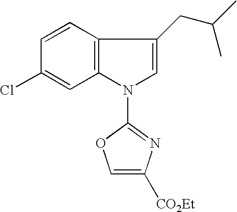 | LCMS Rt = 4.18 min [MH]⁺ 347.1, 349.1 |
| Ethyl 2-[6-chloro-3- (2-methylpropanoyl)- 1H-indol-1-yl]-1,3- thiazole-4- carboxylate (D38) | D1 | 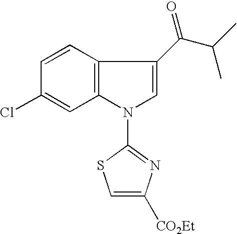 | LCMS Rt = 3.62 min [MH]⁺ 377.19, 379.18 |
| Ethyl 2-[6-chloro-3- (1-methylethyl)-1H- indol-1-yl]-1,3- thiazole-4- carboxylate (D39) | D22 | 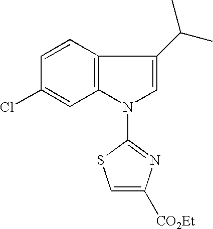 | LCMS Rt = 4.19 min [MH]⁺ 349.16, 351.15 |
| Ethyl 2-[6-chloro-3- (phenylmethyl)-1H- indol-1-yl]-1,3- thiazole-4- carboxylate (D40) | D16 | 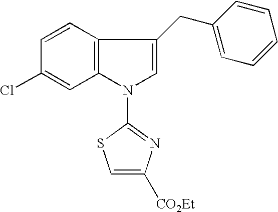 | LCMS Rt = 4.16 min [MH]⁺ 397.19, 399.18 |
| Ethyl 2-[6-chloro-3- (2,2,2-trifluoroethyl)- 1H-indol-1-yl]-1,3- thiazole-4- carboxylate (D41) | D28 | 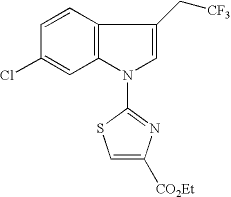 | LCMS Rt = 3.74 min [MH]⁺ 389.14, 391.14 |
| Methyl 3-[6-chloro-3- (2-methylpropyl)-1H- indol-1-yl]benzoate (D42) | D11 | 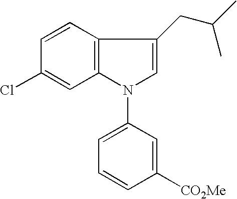 | LCMS Rt = 4.39 min [MH]⁺ 342.25, 344.24 |
| Ethyl 2-[6-chloro-3- (2-methylpropyl)-1H- indazol-1-yl]-1,3- thiazole-4- carboxylate (D43) | D31 | 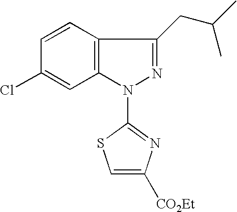 | LCMS Rt = 4.23 min [MH]⁺ 364.17, 366.16 |

### Description 44

### Ethyl (2-bromo-1,3-thiazol-4-yl)acetate (D44)

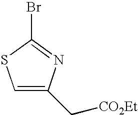

To an ice cooled suspension of ethyl (2-amino-1,3-thiazol-4-yl)acetate (1 g, 5.37 mmol) and copper II bromide (1.44 g, 6.4 mmol) in acetonitrile (6 ml), isoamyl nitrite (1.08 ml, 8 mmol) was added dropwise and the suspension allowed to warm to room temperature with stirring overnight. The solvent was evaporated and the residue dissolved in ethyl acetate/ hexane, filtered and the filtrate evaporated to dryness. The residue was purified by flash chromatography on silica gel eluting with 5% methanol/dichloromethane to give the title compound (420 mg) as a colourless oil.

LCMS: Rt =2.45 min [M+H] 252.0.

### Description 45

### Ethyl 2-[6-fluoro-3-(2-methylpropyl)-1H-indol-1-yl]-1,3-thiazole-4-carboxylate (D45)

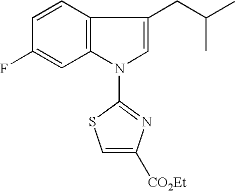

A mixture of 6-fluoro-3-(2-methylpropyl)-1H-indole (may be prepared as described in D17; 971 mg, 5.07 mmol), ethyl 2-bromo-4-thiazolecarboxylate (1 g, 4.23 mmol), potassium phosphate (1.88 g, 8.89 mmol), copper(I) iodide (40 mg, 0.21 mmol) and (1R, 2R)-N,N′-dimethyl-1,2-cyclohexanediamine (120 mg, 0.85 mmol) in toluene (15 ml) was stirred and heated at 110° C. under argon for ˜20 hr. Cooled, filtered trough a pad of silica gel using ethyl acetate to rinse, evaporated, and purified on a silica column using 10% of ethyl acetate in hexane. The residue was triturated with hexane to give the title compound as a pale yellow solid.

LCMS: Rt=4.06 min, [MH⁺] 347.2, 348.2.

### Descriptions 46-50

The following compounds were prepared in an analogous manner to that described in D46 using the appropriate indole and bromo heterocycle:

| | Starting | | |
|---|---|---|---|
| Name | Material | Structure | Data |
| Methyl 5-[6-chloro-3-(2- methylpropyl)-1H-indol-1-yl]- 2-furancarboxylate (D46) | D11 | 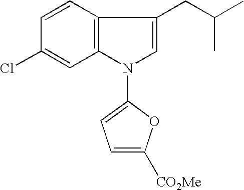 | LCMS Rt = 4.05 min [MH]⁺ 332, 334 |
| Ethyl 6-[6-chloro-3-(2- methylpropyl)-1H-indol-1-yl]- 2-pyridinecarboxylate (D47) | D11 | 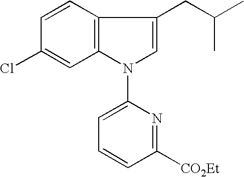 | LCMS Rt = 4.05 min [MH]⁺ 332, 334 |
| Ethyl 2-[6-chloro-3-(2,2- dimethylpropyl)-1H-indol-1- yl]-1,3-thiazole-4-carboxylate (D48) | D18 | 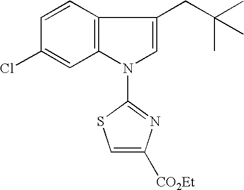 | LCMS Rt = 4.4 min [MH]⁺ 377.2, 379.2 |
| Methyl 2-[6-chloro-3-(2- methylpropyl)-1H-indol-1-yl]- 1,3-thiazole-5-carboxylate (D49) | D11 | 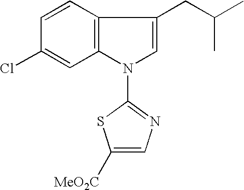 | LCMS Rt = 4.48 min [MH]⁺ 349.1, 351.1 |
| Ethyl {2-[6-chloro-3-(2- methylpropyl)-1H-indol-1-yl]- 1,3-thiazol-4-yl}acetate (D50) | D44 and D11 | 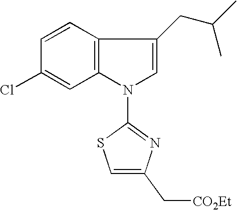 | LCMS Rt = 4.27 min [MH]⁺ 377.1 |

### Description 51

### Ethyl 2-(6-chloro-3-ethyl-1H-indol-1-yl)-1,3-thiazole-4-carboxylate (D51)

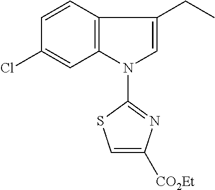

A mixture of 6-chloro-3-ethyl-1H-indole (may be prepared as described in D15; 746 mg, 4.15 mmol), ethyl 2-bromo-4-thiazolecarboxylate (817 mg, 3.46 mmol), potassium phosphate (1.55 g, 7.27 mmol), copper(I) iodide (33 mg, 0.17 mmol) and (1R, 2R)-N,N′-dimethyl-1,2-cyclohexanediamine (98, 0.69 mmol) in toluene (20 ml) was stirred and heated at 110° C. under argon for 4 hr. More copper(I) iodide, (1 R, 2R)-N,N′-dimethyl-1,2-cyclohexanediamine and ethyl 2-bromo-4-thiazolecarboxylate were added and the mixture stirred for a total of 2 days. The solution was cooled, diluted with ethyl acetate/water. The organic phase was washed twice with water, dried (magnesium sulphate), evaporated and purified on the Flash Master II using a gradient of ethyl acetate in hexane. The residue was recrystallised from ether/hexane mixture to give the title compound as a white solid.

LCMS: Rt=4.08 min, [MH⁺] 335.2, 337.2.

### Description 52

### Ethyl 5-[6-chloro-3-(2-methylpropyl)-1H-indol-1-yl]-2-thiophenecarboxylate (D52)

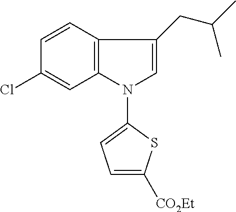

A mixture of 6-chloro-3-(2-methylpropyl)-1H-indole (may be prepared as described in D 1; 414 mg, 2 mmol), ethyl 5-bromothiophene-2-carboxylate (470 mg, 2 mmol), potassium phosphate (850 mg, 4 mmol), copper(I) iodide (19 mg, 0.1 mmol) and (1R, 2R)-N,N′-dimethyl-1,2-cyclohexanediamine (33 mg, 0.23 mmol) in toluene (10 ml) was stirred and heated at 110° C. over night. A further quantity of copper(I) iodide (10 mg, 0.05 mmol) and (1R, 2R)-N,N′-dimethyl-1,2-cyclohexanediamine (1 7 mg, 0.2 mmol) was added. After heating for a further 3 hours the mixture was cooled, diluted with ethyl acetate/water and the organic phase dried, evaporated and purified by flash chromatography eluting with 5% ethyl acetate/hexane to give the title compound as a pink oil (380 mg).

LCMS: Rt=4.44 min [MH]⁺362.18.

### Description 53

### Ethyl 2-[3-(2-methylpropyl)-6-(trifluoromethyl)-1H-indol-1-yl]-1,3-thiazole-4-carboxylate (D53)

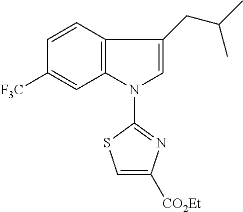

D53 was prepared from D19 in an analogous manner to that described for D52.

### Description 54

### Ethyl 2-{[6-chloro-3-(2-methylpropyl)-1H-indol-1-yl]methyl}-1,3-thiazole-4-carboxylate (D54)

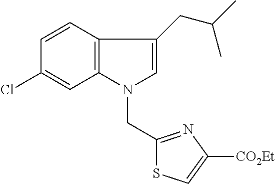

60% Sodium hydride (100 mg, 2.5 mmol) was added to a stirred solution of 6-chloro-3-(2-methylpropyl)-1H-indole (may be prepared as described in D11; 519 mg, 2.5 mmol) in dimethylformamide (8 ml) under argon and stirred for 10 minutes when ethyl 2-(bromomethyl)-1,3-thiazole-4-carboxylate (625 mg, 2.5 mmol) were added. The solution was stirred at room temperature for one hour then diluted with ether/water. The organic phase was washed three times with water, dried (magnesium sulphate), evaporated and purified on a Biotage column eluting with (20:80) ethyl acetate/hexane then triturated with hexane to give the title compound as a white solid (388 mg).

LCMS: Rt=3.83 min, [MH⁺] 377.19, 379.18.

### Description 55

### 1-[2-(Ethenylamino)-4-methylphenyl]-3-methyl-2-butanone (D55)

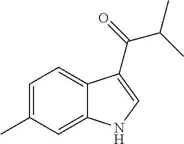

A solution of 6-methyl-1H-indole (0.47 ml, 3.81 mmol) in Et₂O (9.9 ml) was stirred at 0° C. under an atmosphere of argon. A solution of methyl magnesium bromide in Et₂O (3M, 1.29 ml, 3.89 mmol) was added slowly to the reaction mixture over a period of 5 minutes. A slight exotherm was observed. The reaction mixture was stirred for 30 minutes allowing the reaction contents to warm to room temperature. After this time, zinc chloride in Et₂O (1M, 3.81 ml, 3.81 mmol) was added slowly to the reaction mixture. Green precipitate observed. The mixture was stirred at room temperature for a further 15 minutes. After this time, 2-methylpropanoyl chloride (0.40 ml, 3.81 mmol) was added to the reaction mixture. The mixture was stirred for 15 minutes at room temperature. The reaction was monitored by LC-MS. The reaction was quenched by the dropwise addition of saturated ammonium chloride (10 ml). The organics were extracted with EtOAc, dried over magnesium sulfate, filtered and concentrated under reduced pressure to give an orange solid. The crude product was recrystallized from Et₂O to give a white solid, 1-[2-(ethenylamino)-4-methylphenyl]-3-methyl-2-butanone (0.43 g, 56%).

LCMS Rt=2.64 min, [M+H] 202, 203.

### Description 56

### Ethenyl[5-methyl-2-(3-methylbutyl)phenyl]amine (D56)

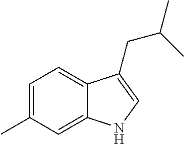

A solution of lithium aluminium hydride in THF (1M, 4.10 ml, 1.94 mmol) was added slowly to a stirred solution of 1-[2-(ethenylamino)-4-methylphenyl]-3-methyl-2-butanone (may be prepared as described in D55; 0.43 g, 2.14 mmol) in dry THF (5 ml) at 0° C. under an atmosphere of argon. Slight effervescence observed. The reaction mixture was left stirring at 0° C. for 30 min under an atmosphere of argon. After this time, the reaction mixture was heated to 50° C. for 90 minutes. The reaction was monitored by LC-MS. The reaction mixture was cooled to 0° C. and quenched by the dropwise addition of water. Colour change of pink to green observed. The solution was stirred at 0° C. for further 10 minutes and then allowed to warm to room temperature. The organics were extracted using EtOAc. The combined organics were dried over magnesium sulfate, filtered and concentrated under reduced pressure to give a brown oil, ethenyl[5-methyl-2-(3-methylbutyl)phenyl]amine (0.27 g, 67%).

LCMS Rt=3.53 min, [M+H] 188,189.

### Description 57

### Ethyl 2-{ethenyl[5-methyl-2-(3-methylbutyl)phenyl]amino}-1,3-thiazole-4-carboxylate (D57)

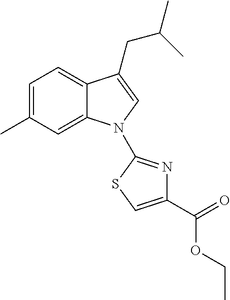

A solution of ethenyl[5-methyl-2-(3-methylbutyl)phenyl]amine (may be prepared as described in D56; 0.27 g, 1.43 mmol), ethyl 2-bromo-1,3-thiazole-4-carboxylate (0.34 g, 1.43 mmol), copper iodide (0.017 g, 0.09 mmol), (1R, 2R) trans-diaminomethylcyclohexane (0.030 g, 0.21 mmol) and potassium phosphate (0.64 g, 3.00 mmol) in PhCH₃ (2.8 ml) was stirred at 110° C. for 16 hours under an atmosphere of argon. The reaction was monitored by LC-MS. After this time, further ethyl 2-bromo-1,3-thiazole-4-carboxylate (0.17 g, 0.72 mmol) was added to the reaction mixture and the mixture stirred at 110° C. for a further 1 hour. After this time, the reaction mixture was allowed to cool to room temperature, and was then partitioned between EtOAc and water. The organics were washed with water, dried over magnesium sulfate, filtered and concentrated under reduced pressure to give brown oil. The residue was purified using column chromatography [SiO₂, Hexane:EtOAc (1:0 to 9:1)] to give ethyl 2-{ethenyl[5-methyl-2-(3-methylbutyl)phenyl]amino}-1,3-thiazole-4-carboxylate (0.059 g, 12%).

LCMS Rt=4.27 min, [M+H] 343, 344.

### Example 1

### 2-(6-Chloro-3-propyl-1H-indol-1-yl)-1,3-thiazole-4-carboxylic acid (E1)

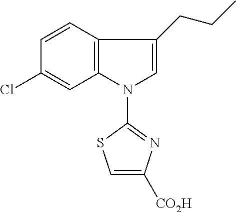

Ethyl 2-(6-chloro-3-propyl-1H-indol-1-yl)-1,3-thiazole-4-carboxylate (may be prepared as described in D33; 80 mg, 0.23 mmol) was dissolved in hot ethanol (5 ml) and 2M sodium hydroxide (1 ml) added and left for 15 minutes. The solution was evaporated to dryness, dissolved in ethyl acetate/2M hydrochloric acid and the organic phase dried (magnesium sulphate), evaporated and triturated with ether to give the title compound as a white solid (54 mg).

LC/MS: Rt=3.35 min, [MH⁺] 321.19, 323.18.

### Examples 2-15

The following compounds were prepared by treating the appropriate ester indicated in the table below with sodium hydroxide using an analogous procedure to that described for E1:

| | Starting | | |
|---|---|---|---|
| Name | Material | Structure | Data |
| 2-[6-Chloro-3-(3,3- dimethylbutyl)-1H-indol- 1-yl]-1,3-thiazole-4- carboxylic acid (E2) | D34 | 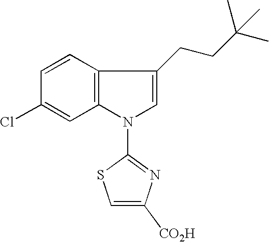 | LCMS Rt = 4.03 min [MH]⁺ 363.18, 365.23 |
| 2-[6-Chloro-3-(1- methylethyl)-1H-indol-1- yl]-1,3-thiazole-4- carboxylic acid (E3) | D39 | 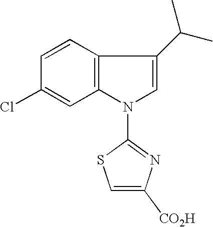 | LCMS Rt = 3.53 min [MH]⁺ 321.15, 323.16 |
| 2-[6-Bromo-3-(2- methylpropyl)-1H-indol-1- yl]-1,3-thiazole-4- carboxylic acid (E4) | D36 | 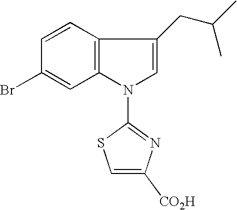 | LCMS Rt = 3.65 min [MH]⁺ 379.10, 382.15 |
| 2-[6-Chloro-3-(2- methylpropyl)-1H-indol-1- yl]-1,3-oxazole-4- carboxylic acid (E5) | D37 | 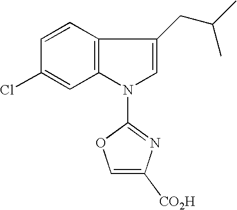 | LCMS Rt = 3.57 min [MH]⁺ 319.20, 321.19 |
| 2-{[6-Chloro-3-(2- methylpropyl)-1H-indol-1- yl]methyl}-1,3-thiazole-4- carboxylic acid (E6) | D54 | 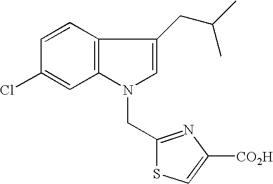 | LCMS Rt = 3.31 min [MH]⁺ 349.23, 351.22 |
| 2-[6-Chloro-3-(2- methylpropanoyl)-1H- indol-1-yl]-1,3-thiazole-4- carboxylic acid (E7) | D38 | 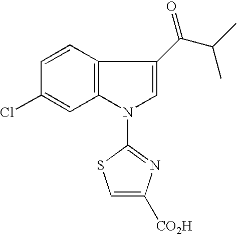 | LCMS Rt = 2.97 min [MH]⁺ 349.15, 351.15 |
| 2-[6-Chloro-3- (phenylmethyl)-1H-indol- 1-yl]-1,3-thiazole-4- carboxylic acid (E8) | D40 | 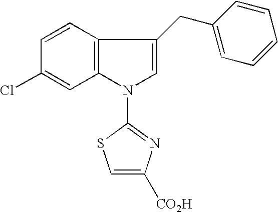 | LCMS Rt = 3.54 min [MH]⁺ 369.15, 371.18 |
| 2-[6-Chloro-3-(2,2,2- trifluoroethyl)-1H-indol-1- yl]-1,3-thiazole-4- carboxylic acid (E9) | D41 | 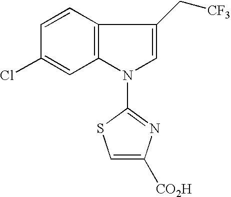 | LCMS Rt = 3.11 min [MH]⁺ 361.11, 363.08 |
| 2-[3-(2-Methylpropyl)-1H- indol-1-yl]-1,3-thiazole-4- carboxylic acid (E10) | D35 | 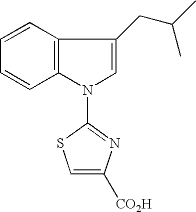 | LCMS Rt = 3.27 min [MH]⁺ 301.27 |
| 2-[6-Chloro-3-(2- methylpropyl)-1H- indazol-1-yl]-1,3-thiazole- 4-carboxylic acid (E11) | D43 | 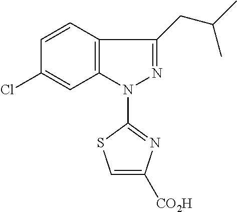 | LCMS Rt = 3.51 min [MH]⁺ 336.20, 338.20 |
| 2-[6-Chloro-3-(2,2- dimethylpropyl)-1H-indol- 1-yl]-1,3-thiazole-4- carboxylic acid (E12) | D48 | 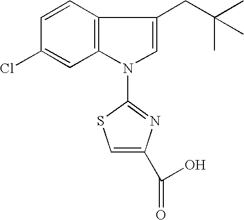 | LCMS Rt = 3.86 min [MH]⁺ 349.2, 351.2, [MH]⁻ 347.15 |
| 2-[3-(2-Methylpropyl)-6- (trifluoromethyl)-1H-indol- 1-yl]-1,3-thiazole-4- carboxylic acid (E13) | D53 | 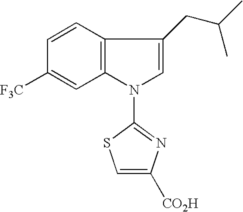 | LCMS Rt = 3.66 min [MH]⁺ 369.1 |
| 3-[6-Chloro-3-(2- methylpropyl)-1H-indol-1- yl]benzoic acid (E14) | D42 | 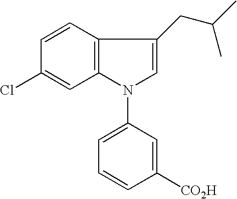 | LCMS Rt = 3.92 min [MH]⁺ 328.30, 330.23 |
| {2-[6-Chloro-3-(2- methylpropyl)-1H-indol-1- yl]-1,3-thiazol-4-yl}acetic acid (E15) | D50 | 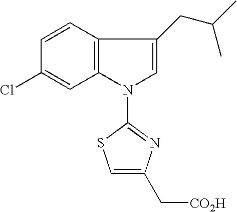 | LCMS Rt = 3.68 min [MH]⁺ 349.1 |

### Example 16

### 2-[6-Fluoro-3-(2-methylpropyl)-1H-indol-1-yl]-1,3-thiazole-4-carboxylic acid (E16)

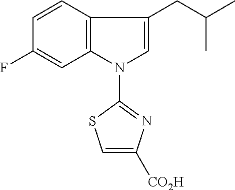

Ethyl 2-[6-fluoro-3-(2-methylpropyl)-1H-indol-1-yl]-1,3-thiazole-4-carboxylate (may be prepared as described in D45; 857 mg, 2.47 mmol) was dissolved in ethanol (8 ml) and 2M sodium hydroxide (3 ml) added and stirred at 50° C. for 2 hr. The solution was cooled, evaporated to dryness, dissolved in ethyl acetate and acidified with 2M hydrochloric acid, extracted with ethyl acetate (x3). The combined organic phase was dried (MgSO₄), evaporated and triturated with hexane/ether to give the title compound as a pale yellow solid (610 mg).

LCMS: Rt=3.41 min, [MH⁺] 319.2, 320.2.

### Example 17

### 2-[6-Chloro-3-(2-methylpropyl)-1H-indol-1-yl]-1,3-thiazole-4-carboxylic acid (E17)

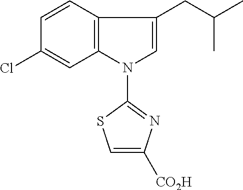

Ethyl 2-[6-chloro-3-(2-methylpropyl)-1H-indol-1-yl]-1,3-thiazole-4-carboxylate (may be prepared as described in D32; 2.6 g not clean) was dissolved in ethanol (20 ml) and 2M sodium hydroxide (10 ml) added and stirred at 50° C. for 1 hr. The solution was cooled, evaporated to dryness, diluted with water and extracted with diethyl ether (x3). The aqueous phase was then acidified with 2M hydrochloric acid, extracted with ethyl acetate (x3), the organic phase was dried (MgSO₄) and evaporated to give the title compound as pale yellow solid (1.5 g)

LCMS: Rt=3.67 min, [MH⁺] 335.1, 337.1, [MH⁻] 333.2, 335.1

### Examples 18-21

The following compounds were prepared from the starting material indicated in the table below in an analogous manner to that described in E17 except that E19 and E20 were only neutralised with hydrochloric acid:

| | Starting | | |
|---|---|---|---|
| Name | Material | Structure | Data |
| 5-[6-Chloro-3-(2- methylpropyl)-1H-indol-1- yl]-2-furancarboxylic acid (E18) | D46 | 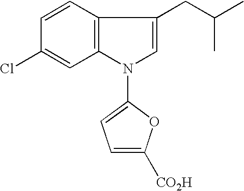 | LCMS Rt = 3.66 min [MH]⁺ 318.1, 320.1, [MH]⁻ 316.2, 318.2 |
| 6-[6-Chloro-3-(2- methylpropyl)-1H-indol-1- yl]-2-pyridinecarboxylic acid (E19) | D47 | 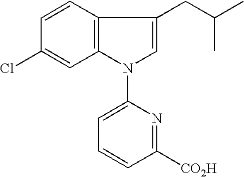 | LCMS Rt = 3.54 min [MH]⁺ 329, 331, [MH]⁻ 327, 329 |
| 2-(6-Chloro-3-ethyl-1H- indol-1-yl)-1,3-thiazole-4- carboxylic acid (E20) | D51 | 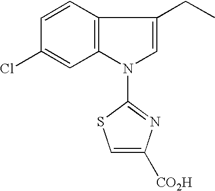 | LCMS Rt = 3.36 min [MH]⁺ 307.2, 309.2, |
| 2-[6-Chloro-3-(2- methylpropyl)-1H-indol-1- yl]-1,3-thiazole-5- carboxylic acid (E21) | D49 | 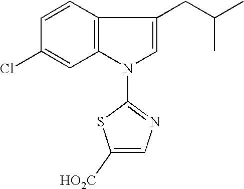 | LCMS Rt = 4.01 min [MH]⁺ 335.1, 337.1 |

### Example 22

### 5-[6-Chloro-3-(2-methylpropyl)-1H-indol-1-yl]-2-thiophenecarboxylic acid (E22)

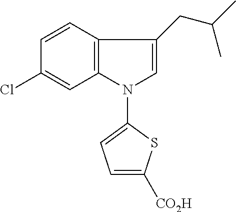

Ethyl 5-[6-chloro-3-(2-methylpropyl)-1H-indol-1-yl]-2-thiophenecarboxylate (may be prepared as described in D52; 500 mg not clean) was dissolved in ethanol (2 ml) and 2M sodium hydroxide (1 ml) added and stirred at room temperature for 1 hr. The solution was cooled, evaporated to dryness, acidified with 2M and extracted with ethyl acetate. The organic phase was dried (MgSO₄), evaporated and purified on MDAP to give the title compound as a white solid (8 mg).

LCMS: Rt=3.84 min, [MH]⁺334.1.

### Example 23

### 2-[6-Chloro-3-(2-methylpropyl)-1H-indol-1-yl]-N-(phenylsulfonyl)-1,3-thiazole-4-carboxamide (E23)

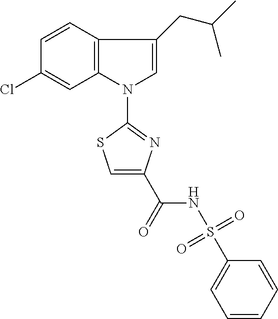

A mixture of 2-[6-chloro-3-(2-methylpropyl)-1H-indol-1-yl]-1,3-thiazole-4-carboxylic acid (may be prepared as described in E17; 112 mg, 0.33 mmol), benzenesulphonamide (63 mg, 0.4 mmol) EDAC (96 mg, 0.5 mmol) and 4-dimethylaminopyridine (2 mg) in dichloromethane (3 ml) was stirred at room temperature for 6 hours then diluted with ethyl acetate and washed twice with 2M sodium hydroxide and once with 2M hydrochloric acid. The organic phase was dried (magnesium sulphate), evaporated and purified on a Biotage column eluting with ethyl acetate/hexane (2:3). The product was triturated with ether to give the title compound as a white solid (53 mg).

LCMS: Rt=4.10 min, [MH⁺] 474.19, 476.18.

### Example 24

### 2-{Ethenyl[5-methyl-2-(3-methylbutyl)phenyl]amino}-1,3-thiazole-4-carboxylic acid (E24)

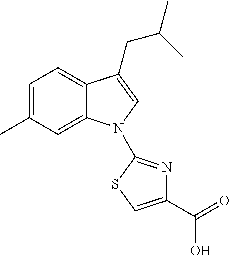

A solution of ethyl 2-{ethenyl[5-methyl-2-(3-methylbutyl)phenyl]amino}-1,3-thiazole-4-carboxylate (may be prepared as described in D57; 0.059 g, 0.17-mmol) and 2M sodium hydroxide (0.36 ml, 0.72 mmol) in EtOH (0.5 ml) was stirred at 90° C. for 1 hour. The reaction was monitored by LC-MS. After this time, the reaction mixture was allowed to cool to room temperature. The reaction mixture was diluted with water and acidified to pH 1 using 2M HCl. The organics were extracted with EtOAc. The combined organics were dried over sodium sulfate, filtered and concentrated under reduced pressure. The residue was triturated using hexane to give a white solid, 2-{ethenyl[5-methyl-2-(3-methylbutyl)phenyl]amino}-1,3-thiazole-4-carboxylic acid (0.02 g, 37%).

LCMS Rt=3.61 min, [M+H] 315.

It is to be understood that the present invention covers all combinations of particular and preferred subgroups described herein above.

Assays for Determining Biological Activity

The compounds of formula (I) can be tested using the following assays to demonstrate their prostanoid antagonist or agonist activity in vitro and in vivo and their selectivity. Prostaglandin receptors that may be investigated are DP, EP₁, EP₂, EP₃, EP₄, FP, IP and TP.

Biological Activity at EP₁ and EP₃ Receptors

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable (pCIN; BioTechniques 20(1996): 102-110) vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing Fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (pIC₅₀) may then be estimated.

Binding Assay for the Human Prostanoid EP₁ Receptor

Competition assay using [³H]-PGE₂

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μpg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5 mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10s burst at full setting), centrifuged at 48,000×g for 20 mins and the pellet containing the membrane fraction is washed (optional) three times by suspension and centrifugation at 48,000×g for 20 mins. The final membrane pellet is suspended in an assay buffer such as 10 mM 2-[N-morpholino]ethanesulphonic acid, 1 mM Na₂EDTA,10 mM MgCl₂ (pH 6). Aliquots are frozen at −80° C. until required.

For the binding assay the cell membranes, competing compounds and [³H]-PGE₂ (3 nM final assay concentrations are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques to determine the concentration of compound producing 50% inhibition of specific binding (IC₅₀).

Results

The compounds of examples 1-24 were tested in the binding assay for the human prostanoid EP₁ receptor. The results are expressed as pIC₅₀values. A pIC₅₀is the negative logarithms of the IC₅₀. The results given are averages of a number of experiments. The compounds of examples 1-24 had a pIC₅₀ value ≧6. More particularly, the compounds of examples 1-6, 11-13, 16-19, and 21-24 exhibited a pIC₅₀ value ≧7.

The compounds of examples 1-24 were tested in the human EP₁ calcium mobilisation assay. The results are expressed as functional pKᵢ values. A functional pKᵢ is the negative logarithms of the antagonist dissociation constant as determined in the human EP₁ calcium mobilisation assay. The results given are averages of a number of experiments. The compounds of examples 1-24 exhibited a functional pKᵢ value ≧6.0. More particularly, the compounds of examples 1-8, 10-13, and 15-24 exhibited a functional pKᵢ value of ≧7.0.

The compounds of examples 1-24 were tested in the human EP₃ calcium mobilisation assay. The results are expressed as functional pKᵢ values. A functional pKᵢ is the negative logarithms of the antagonist dissociation constant as determined in the human EP₃ calcium mobilisation assay. The results given are averages of a number of experiments. The compounds of examples 1-24 exhibited a functional pKᵢ value of ≦6.7. The compounds of examples 7 and 20 exhibited a functional pKᵢ value of ≦5.7.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 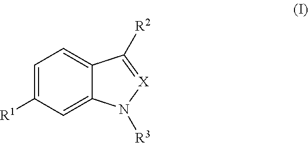
wherein
R¹ represents hydrogen, methyl, —CF₃, chlorine, fluorine or bromine;
R² represents ethyl, propyl, isopropyl, isobutyl, —CH₂-t-butyl, —(CH₂)₂-t-butyl, optionally substituted —CH₂-phenyl, —CH₂—CF₃ or —CO-isopropyl;
X represents CH or N;
R³ represents a group of formula (i)-(ix): 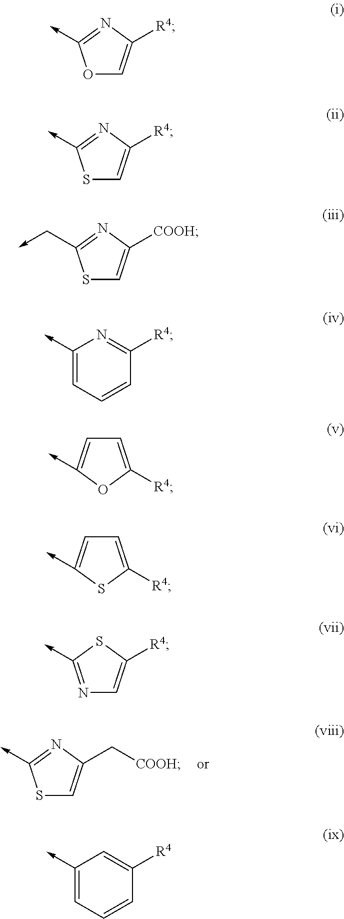
R⁴ represents —COOH, —CO—NH—SO₂—R⁵ or tetrazole;
R⁵ represents C₁₋₃ alkyl, optionally substituted phenyl or 2,4-dimethylisoxazol-4-yl;
such that when R¹ represents hydrogen, R² represents isobutyl;
or derivatives thereof.

2. (canceled)

3. A pharmaceutical composition comprising a compound according to claim 1 or a pharmaceutically acceptable derivative thereof together with a pharmaceutical carrier and/or excipient.

4-5. (canceled)

6. A method of treating a human or animal subject suffering from a condition which is mediated by the action of PGE₂ at EP₁ receptors which comprises administering to said subject an effective amount of a compound according to claim 1 or a pharmaceutically acceptable derivative thereof.

7. A method of treating a human or animal subject suffering from a pain, or an inflammatory, immunological, bone, neurodegenerative or renal disorder, which method comprises administering to said subject an effective amount of a compound according to claim 1 or a pharmaceutically acceptable derivative thereof.

8. A method of treating a human or animal subject suffering from inflammatory pain, neuropathic pain or visceral pain which method comprises administering to said subject an effective amount of a compound according to claim 1 or a pharmaceutically acceptable derivative thereof.

9-11. (canceled)
